(19) United States
(12) Patent Application Publication
(10) Pub. No.: US 2010/0233256 A1
(43) Pub. Date: Sep. 16, 2010

(54) SYNERGISTIC COMBINATIONS FOR TREATING HYPERTENSION

(75) Inventors: Morris Zelkha, Omer (IL); Esther Paran, Omer (IL); Keren Hirsch, Arad (IL)

Correspondence Address: NIXON & VANDERHYE, PC, 901 NORTH GLEBE ROAD, 11TH FLOOR, ARLINGTON, VA, 22203, US

(73) Assignee: LYCORED LTD., Beer Sheva (IL)

(21) Appl. No.: 12/452,619

(22) PCT Filed: Jul. 10, 2008
(86) PCT No.: PCT/IL2008/000956

§ 371 Date: Mar. 26, 2010

(30) Foreign Application Priority Data
Jul. 12, 2007 (IL) 184575

(51) Int. Cl. A61K 9/48; A61K 31/01; A61K 31/19; A61P 9/12; A61K 31/047; A61K 31/56; A61K 31/122

(52) U.S. Cl. 424/456; 514/762; 514/557; 514/729; 514/171; 514/691

(57) ABSTRACT

The present invention relates to methods and compositions effective in lowering blood pressure. The compositions comprise, as active ingredients, an effective amount of a combination of lycopene and a carotenoid, e.g., lutein. In a preferred embodiment, the lycopene and lutein together provide a synergistic therapeutic effect at lowering blood pressure. The compositions can further comprise additional agents such as carotenoids and other phytochemicals found in tomato oleoresin.

### FIELD OF THE INVENTION

The present invention relates to the field of carotenoid containing compositions and uses thereof in treating, preventing and lowering the risk of developing cardiovascular diseases and conditions associated therewith. The compositions are particularly useful in reducing blood pressure.

### BACKGROUND OF THE INVENTION

Carotenoids are naturally occurring pigments found in certain plants and algae. For example, the red color of tomatoes and yellow color of corn derive from carotenoids. Mammals are incapable of synthesizing carotenoids de novo and must obtain them through their diet. High dietary intake of carotenoids has been shown to be associated with reduced incidence of certain types of cancers, cardiovascular disease and hypertension. A dermal photoprotective effect is also associated with a high dietary intake of carotenoids. Lycopene, beta-carotene, phytofluene, phytoene, astaxanthin and cathaxanthin are among the carotenoids which have been found to demonstrate beneficial effects on human health, particularly on the vascular system.

Emerging evidence suggests that diet-derived antioxidants, in particularly carotenoids, may play a role in the prevention of cardiovascular diseases by diminishing the oxidative damage to endothelial cells [1-3]. Tomatoes and their products represent a main source of different classes of compounds with antioxidant properties such as carotenoids, ascorbic acid and a-tocopherol. Furthermore, owing to their high consumption, tomatoes represent a main source of carotenoids in the western diet. Carotenoids reduced the expression of adhesion molecules on cell surface and binding of monocytes to endothelial cells [4]. In addition, inverse relations between carotenoids, vitamin C and inflammatory markers; soluble ICAM, C-reactive protein and leukocytes has been demonstrated [5]. The applicants of the present invention have previously shown that tomato-juice extract containing capsules (Lyc-O-Mato®) meaningfully decrease systolic and diastolic blood pressure in individuals suffering from essential hypertension who does not require medical treatment [6]. The applicants have further demonstrated that addition of Lyc-O-Mato® to treated moderate hypertensives resulted in a significant decrease of their blood pressure, in concordance with elevated lycopene concentration and nitrite level, an NO derivate, in the blood [7]. However the exact site of the antioxidant effect of the tomato juice essence and the mechanism of its blood pressure lowering property is not known.

U.S. Pat. No. 5,705,526 (Fujiwara, et al.) relates to a method for treating hypercholesterolemia by administering to said patient a hypercholesterolemia therapeutic agent containing lycopene as an effective ingredient therein, wherein said lycopene is administered to said patient in an amount within a range of from 1 to 25 mg per day per adult. PCT/IL98/00286 (WO 98/57622) in the name of Lycored Natural Industries Ltd. describes the use of a synergistic mixture of lycopene and vitamin E for inhibiting the oxidation of LDL in human blood, and thus effectively inhibiting the progression of atherosclerosis. Hypertension, which affects a large number of the population, increases the risk of cardiovascular disease. Thus, there is much interest in methods for reducing blood pressure.

There are at least three known categories of methods for decreasing blood pressure in mammals:
1) Category 1: Long term therapy on blood lipid profile-Treating atherosclerosis in order to increase the cross-sectional area of the blood vessel. Said treatment is usually via lowering LDL concentration and modifying LDL/HDL ratio in the blood, and preventing/inhibiting the oxidation of LDL in the blood.
2) Category 2: Treatment of physical properties of the blood (e.g. lowering viscosity and inhibiting/preventing platelet aggregation);
3) Category 3: Treatment of physical/mechanical properties of blood vessels (e.g. flexibility of the arterial wall and vasodilator response).

The effect of category 1 treatment on patients is usually noticeable after longer periods of treatment (e.g. more than 2 weeks), whereas treatment under categories 2 and 3 take effect within shorter periods of time (e.g. 2 days to 3 weeks). Hypercholesterolemia and atherosclerosis which may cause hypertension are treated according to category 1. Therapeutic methods within Categories 2 and 3 employ drugs such as aspirin and vasodilating drugs such as nifedipine. There are undesirable side effects associated with the use of said drugs.

Galley et al, Clinical Science (1997) 92, 361-365, disclose an oral antioxidant synergistic combination comprising beta-carotene, vitamin E, vitamin C and other antioxidants, as effective in lowering blood pressure.

PCT/IL02/00054 (WO 02/058683) in the name of Lycored Natural Industries Ltd. describes compositions and methods for lowering blood pressure comprising an effective amount of a carotenoid selected from lycopene, phytofluene, phytoene, astaxanthin and canthaxanthin or mixtures thereof.

There is a need for a method for lowering blood pressure which is according to categories 2 and 3, which does have said side effects and is based on natural products.

### SUMMARY OF THE INVENTION

The present invention relates to methods and compositions effective in preventing, reducing the risk of developing, or treating cardiovascular diseases and conditions. As demonstrated herein, a combination of tomato extract (Lyc-O-Mato®), which is a mixture of carotenoid and α-tocopherol, with lutein, in near physiologic concentrations, reduced the first phase of inflammatory process: adhesion of neutrophils to the endothelium. These results suggest that combinations of lycopene and lutein are effective at reducing vascular inflammation, treating high blood pressure, and treating and preventing cardiovascular diseases. The compositions comprise, as active ingredients, an effective amount of a combination of lycopene and at least one other carotenoid, e.g., lutein. In a preferred embodiment, the lycopene and lutein together provide a synergistic therapeutic effect at lowering blood pressure. The compositions can further comprise additional agents such as carotenoids and other phytochemicals found in tomato oleoresin.

It has been unexpectedly discovered that the combination of lycopene or a lycopene-containing mixture as described herein, and a carotenoid such as lutein, can provide therapeutically effective anti-hypertensive (i.e., blood pressure lowering) effects. In some embodiments, the effect is synergistic, i.e., the lycopene and the lutein produce a significantly better therapeutic effect than the additive effects achieved by each individual constituent when administered alone at a therapeutic dose. Preferably, the overall effect of the combined therapy after a course of treatment will be significantly better than the effects achieved with a course of each of the therapeutic agents individually. Due to their synergistic anti-hypertensive effects, the compositions of the invention are especially useful for the chemo-prevention of cardiovascular diseases that are associated with, or are caused by, high blood pressure. In a preferred embodiment, the lycopene and lutein are provided at a ratio of about 1:10 to about 10:1, more preferably about 1:5 to about 5:1, and even more preferably about 1:1. Exemplary compositions comprise about 1 mg to about 100 mg each of lycopene and lutein, preferably from about 1 mg to about 20 mg each of lycopene and lutein, and even more preferably about 20 mg lutein and about 15 mg lycopene.

The present invention thus provides, in one embodiment, a method for lowering blood pressure in a subject, comprising administering to said subject a blood pressure lowering effective amount of a composition comprising lycopene and a carotenoid, e.g. lutein. In another embodiment, the present invention provides a pharmaceutical composition for lowering blood pressure in a subject, comprising a blood pressure lowering effective amount of composition comprising lycopene and lutein in a ratio of 1:1.

In yet another embodiment, the present invention relates to the use of a composition comprising lycopene and a carotenoid, e.g., lutein for the preparation of a medicament to for lowering blood pressure.

In another embodiment, the present invention relates to a method of reducing vascular inflammation in a subject, by administering to the subject an anti-inflammatory effective amount of composition comprising lycopene and a carotenoid, e.g., lutein.

In one embodiment, the lycopene and the lutein together provide a synergistic therapeutic effect. The term “synergistic” and its various grammatical variations means that the observed effect (e.g., lowering blood pressure) is higher than the sum of the individual effects of each component administered separately. In one embodiment, the observed combined effect of the agents is significantly higher than the sum of the individual effects. The term significant means that the observed p<0.05.

Lycopene can be synthetically prepared or extracted from natural sources. Preferably the lycopene is provided in the composition as a natural compound. In some embodiments, lycopene is provided as an extract, example, as an extract of tomato oleoresin, such as Lyc-O-Mato®. In some embodiments, the oleoresin further comprises at least one agent selected from the group consisting of tocopherols, beta-carotene, phytoene, phytofluene, phytosterols, tomato oil, and phospholipids.

In some embodiments the compositions can further comprise one or more additional phytochemicals, such as zeaxanthin, astaxanthin and canthaxanthin, phytosterols, and adjuvants such as phytoene, phytofluene and carnosic acid or derivatives thereof (e.g., carnosol, 6,7-dehydrocarnosic acid or 7-ketocarnosic acid). If provided, these phytochemical(s), independently of each other, can be separately added to the composition, or they can be present as part of a natural extract, e.g., a lycopene extract. In some embodiments the phytochemicals are synthetically prepared.

In one embodiment, the composition of the invention is administered in conjunction with a conventional anti-hypertensive agent. Aspirin and vasodilating drugs such as nifedipine are non-limiting examples of “conventional anti-hypertensive agents”.

According to another aspect, the compositions of the invention are formulated for oral use in a form selected from a tablet, caplet, capsule, microcapsule, pellet, pill, powder, syrup, gel, slurry, granule, suspension, dispersion, emulsion, liquid, solution, dragee, bead and beadlet and the like. A beadlet is a polysaccharide complex, in the shape of a bead, in which small droplets containing the active material are embedded. In certain preferred embodiments the composition is formulated as a soft gelatin capsule or as a hard gelatin capsule. In other embodiments the composition is formulated as a beadlet based on alginates, gelatin or other natural or synthetic polymers.

In yet a further aspect of the invention, lycopene and lutein, alone or in combination with other carotenoids or adjuvants as herein described, are added to functional foods, dietary supplements or drinks in order to lower blood pressure. The compositions can also be dispensed as dry formulation, for example as powder, granules, microcapsules or capsules, for reconstitution as a liquid, dispersion, emulsion or suspension.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1—The effect of combination of lutein and oleoresin (oleo) or lutein and lycopene 610 (lyc) on the inhibition of adhesion of neutrophils to EA.hy 926.

### DETAILED DESCRIPTION OF THE PRESENT INVENTION

The following description is illustrative of embodiments of the invention. The following description is not to be construed as limiting, it being understood that the skilled person may carry out many obvious variations to the invention.

The present invention relates to methods and compositions effective in treating and preventing cardiovascular diseases, e.g., by lowering blood pressure. Hypertension, or high blood pressure, is known increase the risk of developing cardiovascular diseases. Therefore, the compositions of the invention are useful in treating and preventing cardiovascular diseases in individuals suffering from high blood pressure, that are at risk for developing cardiovascular related conditions. The compositions of the invention are also effective at reducing vascular inflammation.

The compositions comprise, as active ingredients, an effective amount of a combination of lycopene and a carotenoid, preferably lutein. In a preferred embodiment, the lycopene and lutein together provide a synergistic therapeutic effect at lowering blood pressure. The compositions can further comprise additional agents such as carotenoids and other phytochemicals found in tomato oleoresin. It has been unexpectedly discovered that the combination of lycopene or a lycopene-containing mixture as described herein, and lutein, can provide therapeutically effective anti-hypertensive (i.e., blood pressure lowering) effects. In some embodiments, the effect is synergistic, i.e., the lycopene and the lutein produce a significantly better therapeutic effect than the additive effects achieved by each individual constituent when administered alone at a therapeutic dose.

Lycopene

Lycopene is the major carotenoid present in the diet and provides the familiar red color of tomato products. More than 80% of dietary intake of lycopene is derived from tomato sources, such as ketchup, tomato juice, spaghetti sauce, tomato soup and pizza sauce. Lycopene can be prepared synthetically, or can be obtained as a natural tomato extract. For example, lycopene is obtainable under the name Lyc-O-Mato® (LycoRed, Israel) as an all-natural antioxidant formula containing tomato lycopene, tocopherols, beta-carotene, phytoene, phytofluene, phytosterols, tomato oil, phospholipids, and other important bioactive phytochemicals naturally occurring in tomato oleoresin.

According to one exemplary embodiment, lycopene is provided in the present composition in a concentration range of about 0.025% w/w to about 5% w/w. A range of about 0.25% w/w to about 2.5% w/w is preferred, and a concentration of about 1.5% w/w is more preferred.

Lutein

Lutein and its stereoisomer zeaxanthin belong to the xanthophyll family of carotenoids. Green leafy vegetables are the best dietary source of lutein, with spinach, kale and parsley providing high levels. Purified crystalline lutein has been classified generally recognized as safe (GRAS) and can be added to food and beverages. Pure lutein may also be isolated from certain plants, as described, for example, in U.S. Pat. No. 5,382,714 and U.S. Pat. No. 5,648,564.

In one exemplary embodiment, the compositions of the invention comprise lutein. In some embodiments, lutein is present in the composition in a concentration range of about 0.025% w/w to about 5% w/w. A range of about 0.25% w/w to about 2.5% w/w is preferred, and a concentration of about 2% w/w is more preferred. In some embodiments lutein is synthetically prepared. In other embodiments lutein is isolated and purified from a natural source.

According to one exemplary embodiment, each of lycopene and lutein is provided in the present composition in a concentration range of about 0.025% w/w to about 5% w/w. A range of about 0.25% w/w to about 2.5% w/w is preferred, and a concentration of about 1.5% or 2% w/w is more preferred.

According to a particular embodiment of the method of the present invention, about 0.1 to 100 mg per day of each of lycopene and lutein are administered to a subject in need of lowering blood pressure. For the purpose of illustration and not for limitation, each of lycopene and lutein can be provided in an amount of about 1, 2, 3, 4, 5, 6, 7, 8, 9, 10, 15, 20, 25, 30, 35, 40, 50, 60, 70, 80, 90 or 100 mg. In a preferred embodiment, the lycopene and lutein are provided at a ratio of about 1:10 to about 10:1, more preferably about 1:5 to about 5:1, more preferably from about 1:2 to about 2:1, and even more preferably about 1:1 based on a weight/weight ratio. In another embodiment lycopene is present in an amount of about 15 mg, and lutein is present in an amount of about 20 mg.

The administration is preferably oral administration, however, all forms of administration which achieve a blood pressure lowering effective concentration in the blood, e.g. about 0.3 to 0.8 μM of each of lycopene and lutein, is suitable for the purposes of this invention. Administration may be by a single daily dose or multiple doses.

According to a particular embodiment of the method of the present invention, about 0.1 to 50 mg per day in total of a combination of lycopene and lutein are administered to a subject in need of lowering blood pressure. The administration is preferably oral administration, however, all forms of administration which achieve a blood pressure lowering effective concentration in the blood, e.g. about 0.3 to 0.8 μM of each of lycopene and lutein, is suitable for the purposes of this invention. Administration may be by a single daily dose or multiple doses.

Carotenoids

In one currently preferred embodiment, the composition further comprises at least one carotenoid other than lycopene and lutein. Carotenoids useful in the compositions and methods of the present invention can be naturally occurring carotenoids found in, for example, tomato products (e.g., tomatoes, tomato sauce, ketchup and the like), fermentation, watermelon, guava, grapefruit, and other fruits and vegetables containing these carotenoids. The carotenoids can be obtained by extracting the carotenoid from a natural source, using extraction techniques known to a person of skill in the art, or they can be synthetically prepared using any total or semi-synthesis known to a person of skill in the art. Also contemplated are carotenoids derived from genetically modified organisms.

Examples of carotenoids include but are not limited to α-carotene, β-carotene, zeta-carotene, α-cryptoxanthin, β-cryptoxanthin, phytoene, phytofluene, zeaxanthin, astaxantin, canthaxanthin, and combinations thereof.

If provided, these carotenoids can, independently of each other, be separately added to the composition, or they can be present as part of a natural extract, e.g., a lycopene extract. In some embodiments, the carotenoids are synthetically prepared.

In another exemplary embodiment, the compositions of the invention comprise zeaxanthin, a stereoisomer of lutein. In one exemplary embodiment, zeaxanthin is present in the composition in a concentration range of about 0.001% w/w to about 2% w/w. A range of about 0.025% w/w to about 1% w/w is preferred, and a concentration of about 0.5% w/w is more preferred. In some embodiments zeaxanthin is synthetically prepared. In other embodiments zeaxanthin is isolated and purified from a natural source.

In other embodiments, zeaxanthin is contained in the lutein fraction. In accordance with these embodiments, the total concentration of lutein and zeaxanthin is about 0.035% to about 7% w/w, preferably in the range of about 1.5% to about 2.5% w/w, more preferably about 2% to about 2.2% w/w total lutein and zeaxanthin.

Natural sources containing the carotenoids include various fruits and vegetables, as well as various animal products. Lycopene, phytoene and phytofluene are found in tomatoes. Beta carotene (provitamin A), is the carotenoid that gives carrots their orange color, and is converted to vitamin A in the walls of the small intestine (intestinal mucosa) in a reaction catalysed by the enzyme beta-carotene dioxygenase. Alpha carotene is also found in carrots and mixed vegetables. Beta-Cryptoxanthin, also known as cryptoxanthin, cryptoxanthol, and hydroxy-beta-carotene belongs to the xanthophylls class of carotenoids, and is also considered a pro-vitamin A since it can be converted to vitamin A in the human body. Beta-cryptoxanthin can be found in many vegetables and fruits, mainly in papaya, mango, peaches, oranges, tangerines, bell peppers, corn and watermelon. Beta-cryptoxanthin is also found in some yellow colored animal products such as egg yolk and butter. Astaxanthin is found in orange vegetables and in dark leafy greens. It can also be found in seafood such as salmon, trout, red seabream, shrimp, lobster and fish eggs. Canthaxanthin is known mainly as the natural pigment of the orange-yellow Chanterelle mushroom, but also occurs in various lower animals, some crustaceans, insects, fishes and birds. Spinach, kale, collard greens, romaine lettuce, leeks, peas, and egg yolks are good sources of lutein. Sources of zeaxanthin include corn, spinach, collards, oranges and other citrus products, lettuce, peas, beans, broccoli, celery, peaches and carrots.

A single carotenoid as well as combinations and mixtures thereof can be administered in the methods of the present invention. Accordingly, carotenoid mixtures of lycopene and phytoene; lycopene and phytofluene; and lycopene, phytoene, and phytofluene can be administered in the presently claimed methods and compositions.

Adjuvants

In some embodiments, the compositions can further comprise adjuvants such as phytoene, phytofluene and carnosic acid or derivatives thereof. Carnosic acid is an antioxidant extracted from rosemary (Rosemarinus spp) and other herbs, which has been shown to inhibit LDL oxidation in a synergistic manner with lycopene. Phytoene and phytofluene are carotenoids found in tomatoes, and may be found in tomato oleoresin.

If provided, these phytochemical(s) can, independently of each other, be separately added to the composition, or they can be present as part of a natural extract, e.g., a lycopene extract. In some embodiments the phytochemicals are synthetically prepared. In other embodiments the phytochemicals are purified from a natural source.

In one embodiment, phytoene and phytofluene are provided as adjuvants in the present compositions. In an exemplary embodiment, phytoene and phytofluene are present at about 10% of the concentration of lycopene. In another exemplary embodiment, each of phytoene and phytofluene are provided in a concentration range of about 0.0025% w/w to about 1.25% w/w. A range of about 0.025% w/w to about 0.25% w/w of each phytochemical is preferred, and a concentration of 0.15% w/w total of both phytoene and phytofluene, corresponding to about 10% of the lycopene content, is more preferred.

In other embodiments, camosic acid or derivatives thereof, (e.g., camosol, 6,7-dehydrocarnosic acid or 7-ketocarnosic acid) is also provided as an adjuvant in the present compositions. Carnosic acid or its derivatives is preferably provided in a concentration range of about 0.025% w/w to about 2.5% w/w. A range of about 0.25% w/w to about 2% w/w is preferred, and a concentration of 1% w/w is more preferred

### Supplements

Additional agents, including minerals, vitamins, and other micronutrients can optionally be incorporated in the compositions of the present invention.

In some embodiments the composition further comprises a source of zinc, which can be zinc oxide or a zinc salt. Any zinc salt is acceptable. Examples of zinc salts include but are not limited to zinc chloride, zinc acetate, zinc gluconate, zinc carbonate, zinc sulfate, zinc borate, zinc nitrate and zinc silicate. In some embodiments a concentration of about 0.25% to about 15% w/w zinc is provided. In preferred embodiments a concentration of about 7.5% w/w zinc is provided; preferably in the form of zinc gluconate.

In certain embodiments a source of copper is provided in the present composition. In some embodiments cupric oxide is preferred. In some embodiments a copper is provided at a concentration of about 0.01% to about 5% w/w; preferably at a concentration of about 0.25% w/w.

Certain preferred vitamins include vitamin A, vitamin C and vitamin E. The vitamins can be provided as natural or synthetically produced compounds. Derivatives, including provitamins of the preferred vitamins are acceptable. In some preferred embodiments vitamin A is provided as provitamin A, specifically β-carotene. In some embodiments, combinations of the above vitamins are included in the composition. The addition of vitamin E, vitamin C and β-carotene is preferred. Vitamin E and vitamin C can each be included in the present composition at a concentration of about 0.25% to about 25% w/w. Preferable concentrations of vitamin E and vitamin C are about 12.5%. β-carotene can be included in the present composition at a concentration of about 0.025% to 2.5% w/w. Preferable concentrations of β-carotene are about 0.75% w/w.

### Therapeutic Use

High blood pressure, termed “hypertension,” is a condition that afflicts almost 1 billion people worldwide and is a leading cause of morbidity and mortality. More than 20% of Americans are hypertensive, and one-third of these Americans are not even aware they are hypertensive. This disease is usually asymptomatic until the damaging effects of hypertension (such as stroke, myocardial infarction, renal dysfunction, visual problems, etc.) are observed. Data from observational studies involving more than 1 million individuals have indicated that death from ischemic heart disease and stroke increases progressively and linearly from blood pressure levels of 115 mmHg systolic and 75 mmHg diastolic in all age groups. For every 20 mmHg systolic and 10 mmHg diastolic increase in blood pressure there is a doubling of mortality. However, in order to simplify the diagnostic and treatment approaches in the daily practice, a cutoff value of 140/90 had been determined. Recent guidelines (JNC 7 2003 and 2007 guidelines of ESH and ESC) emphasize the importance of isolated systolic HT in contrast to the diastolic values that were considered more important in the past. Clinical studies have demonstrated that control of isolated systolic hypertension reduces total mortality, CV mortality, stroke and heart failure

Most population based studies confirm that even modest increase in blood pressure to high normal values increases an individual's risk of various cardiovascular consequences approximately two to three times. Therefore the term “prehypertension” (JNC7) and high added risk (ESH/ESC) has been introduced as a new target for treatment and prevention of hypertension and its consequences.

As contemplated herein, the present invention provides powerful methods for lowering blood pressure in a subject, by administering a combination of lycopene (either alone or as part of an extract, matrix or composition), and a carotenoid such as lutein. The agents preferably provide a combination which provides a synergistic therapeutic effect at treating blood pressure. By lowering blood pressure, the compositions of the invention are useful in the chemoprevention and treatment of cardiovascular diseases and conditions.

### Pharmaceutical Compositions

Although the active agents lycopene and carotenoid (e.g., lutein), optionally in combinations with other carotenoids, adjuvants and/or supplements, can be administered alone, it is contemplated that these compounds will be administered in a pharmaceutical composition containing the active ingredients together with a pharmaceutically acceptable carrier or excipient.

Thus, a further embodiment of the instant invention is pharmaceutical composition in unit dosage form, useful for lowering blood pressure, the compositions comprising lutein and lycopene; and a pharmaceutically acceptable carrier or excipient. Optionally, the composition further comprises at least one carotenoid such as zeaxanthin, at least one adjuvant such as phytoene, phytofluene and carnosic acid or derivatives thereof. The compositions can also comprise minerals such as zinc and copper, and/or vitamins such as vitamin A (e.g., beta carotene), vitamin C and vitamin E.

Lycopene, lutein and additional agent(s), if provided, can be administered separately or together. It is understood when the compounds are administered separately, administration can be simultaneous or sequential, in any order. For example, lycopene can be administered followed by lutein. Alternatively, lutein can be administered prior to lycopene. Alternatively, the different components of the combination can be administered together, but in separate dosage forms. Alternatively, the different compounds can be administered together in the same pharmaceutical composition. Further, if more than one of the aforementioned classes of compounds is administered, each component can be administered together or apart from the other. For example if two or more carotenoids are administered, they can be administered in separate dosage forms, simultaneously or sequentially, in any order, or they can be provided in the same pharmaceutical composition, for concurrent administration.

Pharmaceutical compositions for use in accordance with the present invention can be formulated in conventional manner using one or more physiologically acceptable carriers comprising excipients and auxiliaries, which facilitate processing of the active compounds into preparations which can be used pharmaceutically. The active agents are formulated as pharmaceutical compositions and administered to a mammalian subject, such as a human patient in a variety of forms such as liquid, solid, and semisolid. The pharmaceutical compositions can be administered to a subject by any method known to a person skilled in the art, such as orally, topically, parenterally, paracancerally, transmucosally, transdermally, intramuscularly, intravenously, intradermally, subcutaneously, intraperitonealy, intraventricularly, intracranially or intratumorally.

For oral administration, the compounds can be formulated by combining the active compounds with pharmaceutically acceptable carriers known in the art. The compositions can be formulated in any solid or liquid dosage form known in the art, including but not limited to, tablet, caplet, capsule, microcapsule, pellet, pill, powder, syrup, gel, slurry, granule, suspension, dispersion, emulsion, liquid, solution, dragee, bead and beadlet. The oral compositions can be formulated as immediate release formulations, or as controlled or sustained release formulations allowing for extended release of the active ingredient(s) over a predetermined time period. Other suitable formulations are those disclosed in U.S. Pat. No. 6,086,915 to Zeligs et al., the contents of which are incorporated by reference herein, are also contemplated.

Suitable excipients for solid formulations include but are not limited to fillers such as sugars, including lactose, sucrose, mannitol, or sorbitol; starch based excipients such as maize starch, wheat starch, rice starch, potato starch and the like, gelatin, gum tragacanth, cellulose based excipients as microcrystalline cellulose, carboxymethylcellulose, hydroxymethylcellulose, hydroxyethylcellulose, methylhydroxypropylcellulose, hydroxypropylcellulose and the like. Polymers such as polyvinylpyrrolidone (PVP) and cross-lined PVP can also be used. In addition, the compositions may further comprise binders (e.g. acacia, cornstarch, gelatin, carbomer, ethyl cellulose, guar gum, hydroxypropyl cellulose, hydroxypropyl methyl cellulose, povidone), disintegrating agents (e.g. cornstarch, potato starch, alginic acid, silicon dioxide, croscarmelose sodium, crospovidone, guar gum, sodium starch glycolate), surfactants (e.g. sodium lauryl sulfate), and lubricants (e.g. stearic acid, magnesium stearate, polyethylene glycol, sodium lauryl sulfate).

For liquid formulations, pharmaceutically acceptable carriers may be aqueous or non-aqueous solutions, suspensions, emulsions or oils. Examples of non-aqueous solvents are propylene glycol, polyethylene glycol, and injectable organic esters. Aqueous carriers include water, alcoholic/aqueous solutions, emulsions or suspensions, including saline and buffered media. Examples of oils include but are not limited to petroleum, animal, vegetable, or synthetic origin, for example, peanut oil, soybean oil, mineral oil, olive oil, sunflower oil, and fish-liver oil.

Preferred oral pharmaceutical compositions include capsules made of gelatin as well as soft, sealed capsules made of gelatin and a plasticizer, such as glycerol or sorbitol. In soft capsules, the active compounds may be dissolved or suspended in suitable liquids, such as fatty oils, liquid paraffin, or liquid polyethylene glycols. In addition, stabilizers may be added. In certain preferred embodiments the capsules exclude components of animal origin and are acceptable for vegetarians and vegans.

Soft gelatin capsules and methods of preparing them are known in the art. Non-limiting examples can be found in U.S. Pat. Nos. 6,217,902; 6,258,380; 5,916,591, and 4,891,229, all of which are incorporated herein by reference.

Other acceptable excipients and additives known to the person with skill in the art may be included in the compositions of the present invention, for example stabilizers, solubilizers, tonicity enhancing agents, buffer substances, preservatives, thickeners, complexing agents and other excipients, as well as additional therapeutic agents.

A solubilizer can be for example, tyloxapol, fatty acid glycerol polyethylene glycol esters, fatty acid polyethylene glycol esters, polyethylene glycols, glycerol ethers or mixtures of those compounds. A specific example of a solubilizer is a polyoxyethylated castor oil for example, the commercial products Cremophor® or Cremophor® RH40. Another example of a solubilizer is tyloxapol. The concentration used depends especially on the concentration of the active ingredient. The amount added is typically sufficient to solubilize the active ingredient. For example, the concentration of the solubilizer is from 0.1 to 5000 times the concentration of the active ingredient.

Examples of buffer substances are acetate, ascorbate, borate, hydrogen carbonate/carbonate, citrate, gluconate, lactate, phosphate, propionate and TRIS (tromethamine) buffers. The amount of buffer substance added is, for example, that necessary to ensure and maintain a physiologically tolerable pH range. The pH range is typically in the range of from 5 to 9, preferably from 5.2 to 8.5.

Tonicity enhancing agents are selected from ionic and non-ionic agents. For example, ionic compounds, include alkali metal or alkaline earth metal halides, such as, for example, CaCl₂ KBr, KCl, LiCl, NaI, NaBr or NaCl, or boric acid. Non-ionic tonicity enhancing agents are, for example, urea, glycerol, sorbitol, mannitol, propylene glycol, or dextrose. For example, sufficient tonicity enhancing agent is added to impart to the ready-for-use ophthalmic composition an osmolality of approximately from 50 to 1000 mOsmol.

Examples of preservatives are quaternary ammonium salts such as benzalkonium chloride, benzoxonium chloride or polymeric quaternary ammonium salts, alkyl-mercury salts of thiosalicylic acid, such as, for example, thiomersal, phenylmercuric nitrate, phenylmercuric acetate or phenylmercuric borate, parabens, such as, for example; methylparaben or propylparaben, alcohols, such as, for example, chlorobutanol, benzyl alcohol or phenyl ethanol, guanidine derivatives, such as, for example, chlorohexidine or polyhexamethylene biguanide, or sorbic acid. Where appropriate, a sufficient amount of preservative is added to the ophthalmic composition to ensure protection against secondary contaminations during use caused by microbes.

The compositions of the present invention may comprise further non-toxic excipients, such as, for example, emulsifiers, wetting agents or fillers, such as, for example, the polyethylene glycols (PEG200, 300, 400 and 600) or Carbowax (Carbowax1000, 1500, 4000, 6000 and 10000). Other excipients that may be used if desired are listed below but they are not intended to limit in any way the scope of the possible excipients. They can be complexing agents, such as disodium-EDTA or EDTA, antioxidants, such as ascorbic acid, acetylcysteine, cysteine, sodium hydrogen sulfite, butyl-hydroxyanisole, butyl-hydroxytoluene; stabilizers, such thiourea, thiosorbitol, sodium dioctyl sulfosuccinate or monothioglycerol; or other excipients, such as, for example, lauric acid sorbitol ester, triethanol amine oleate or palmitic acid ester.

Optionally, the suspension may also contain suitable stabilizers or agents, which increase the solubility of the compounds, to allow for the preparation of concentrated solutions. For example, U.S. Pat. No. 5,576,311, the contents of which are incorporated by reference herein, teaches stable aqueous suspension of drugs comprising cyclodextrin type-suspending agents. Other useful formulations include submicron ocular emulsions, for example an ocular drug delivery vehicle as disclosed in U.S. Pat. No. 5,496,811, the contents of which are incorporated by reference as if fully set forth herein.

The amount and type of excipient added is in accordance with the particular requirements and is generally in the range of from approximately 0.0001 to approximately 90% by weight.

The amount of a composition to be administered will, of course, depend on many factors including the subject being treated, the severity of the affliction, the manner of administration, and the judgment of the prescribing physician. However, the dose employed will generally depend on a number of factors, including the age and sex of the patient, and the severity of the disease being treated.

Preferably, the preparations are in unit dosage form, intended for oral administration. In such form, the preparation is subdivided into unit doses containing appropriate quantities of the active components. The unit dosage form can be a packaged preparation, the package containing discrete quantities of preparation, for example, tablets, capsules, and powders in vials or ampoules. The unit dosage form can also be a capsule, cachet, or tablet itself or it can be the appropriate number of any of these in packaged form.

The dosing schedule of the compositions of the present invention can vary according to the particular application and the potency of the active ingredients. Determination of the proper dosage is within the skill of the art. For convenience, a single daily dose is preferred. Alternatively, the total daily dosage may be divided and administered in portions during the day such as twice daily, thrice daily and the like. Biweekly, weekly, bimonthly and monthly administration are also contemplated

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXPERIMENTAL DETAILS SECTION

### Example 1

### Effect of Lycopene and Lutein on Adhesion of Neutrophils to Endothelial Cells

Increased adhesion of neutrophils is one of the first steps in the intravascular inflammatory process. Neutrophils and their high content of NAD(P)H oxidase have a dominant role in production of vascular ROS (reactive oxidative species). These free oxidative radicals interact with the vascular NO and decrease its level and effect as the major vasodilating substance in the vascular wall. Vascular oxidative stress has been demonstrated in spontaneous and experimental hypertensive animals. Clinical studies demonstrated increased ROS production in patients with essential hypertension and decreased levels of NO. Furthermore any intervention that decreased the oxidative stress or increased the bioavailability of NO decreased the level of BP in hypertensive patients.

The applicants of the present invention have shown that lycopene, lutein and 7% Lye-O-Mato® can improve endothelial function by reducing, to similar extent (60±15%), the adhesion of neutrophils to cytokine-induced endothelial cell line (EAhy. 926). However, these effects were observed with high concentrations of these compounds: lutein 4-5 μM, lycopene 610 2-3 μM, and Lyc-O-Mato® 1-2 μM lycopene. Since the concentration of carotenoids in the serum are <1 μM, the applicants tested the hypothesis that the combination of physiologically concentration of these compound will synergistically inhibit the neutrophils adhesion to EA.hy 926 endothelial cell line.

Lycopene 610 and oleoresin were diluted by 1:12 fold and lutein by 1:8 fold which result in ˜0.2 μM lycopene 610, ˜0.125 μM oleoresin and ˜0.6 μM lutein.

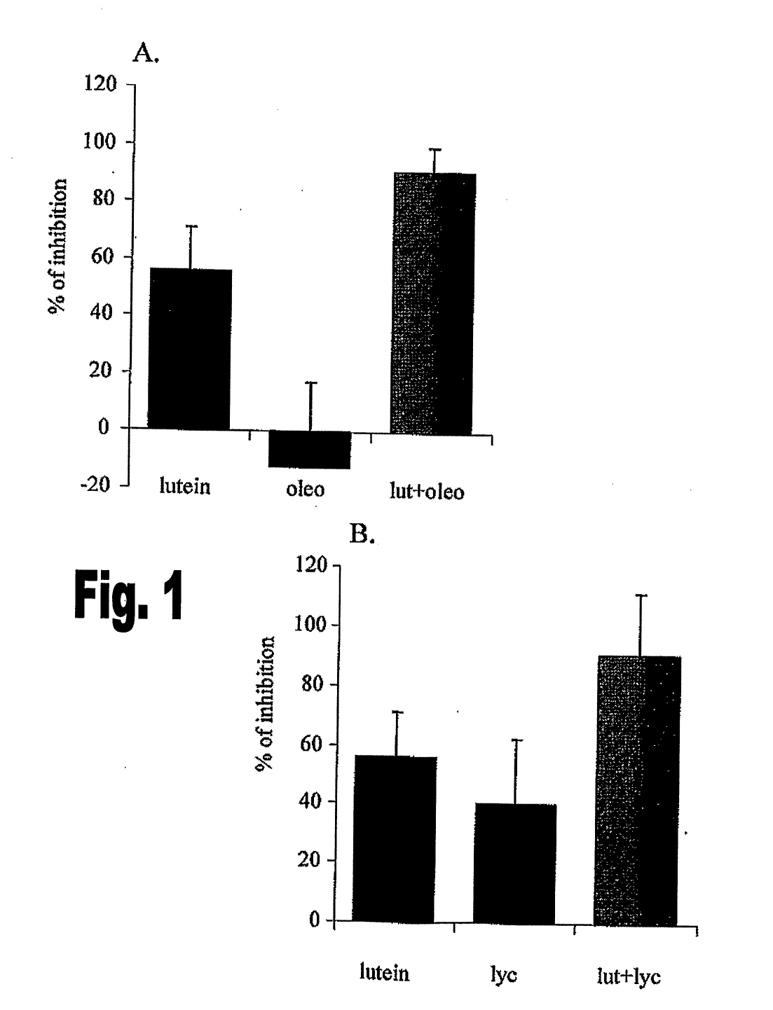

The inhibition of the adhesion with the lower concentration of lutein and oleoresin was 56±15% and −12±30%, respectively, and their combination result in synergistic effect 91±9% (FIG. 1A). However the inhibition by lower concentration of lycopene was 40±22% and the combination with lutein resulted in only additive inhibition (91±21%—FIG. 1B).

The adhesion of neutrophils to EA.hy 926 cells were measured after the endothelial cells were induced by TNFα which result in increase of 60±10% in the adhesion from their combination, was calculated as percent of the adhesion induced by TNFα

The results demonstrate that the combination of tomato extract (Lyc-O-Mato®), which is a mixture of carotenoid and α-tocopherol, with lutein, in near physiologic concentrations, reduced the first phase of inflammatory process: adhesion of neutrophils to the endothelium.

These results suggest that combinations of lycopene and lutein are effective at treating high blood pressure and for treating and preventing cardiovascular diseases.

While certain embodiments of the invention have been illustrated and described, it will be clear that the invention is not limited to the embodiments described herein. Numerous modifications, changes, variations, substitutions and equivalents will be apparent to those skilled in the art without departing from the spirit and scope of the present invention as described by the claims, which follow.

### REFERENCES

1. Gey, K. F., H. B. Stahelin, and M. Eichholzer, Poor plasma status of carotene and vitamin C is associated with higher mortality from ischemic heart disease and stroke: Basel Prospective Study. Clin Investig, 1993. 71(1): p. 3-6.
2. Kardinaal, A. F., et al., Antioxidants in adipose tissue and risk of myocardial infarction: the EURAMIC Study. Lancet, 1993. 342(8884): p. 1379-84.
3. Street, D. A., et al., Serum antioxidants and myocardial infarction. Are low levels of carotenoids and alpha-tocopherol risk factors for myocardial infarction? Circulation, 1994. 90(3): p. 1154-61.
4. Martin, K. R., D. Wu, and M. Meydani, The effect of carotenoids on the expression of cell surface adhesion molecules and binding of monocytes to human aortic endothelial cells. Atherosclerosis, 2000. 150(2): p. 265-74.
5. van Herpen-Broekmans, W. M., et al., Serum carotenoids and vitamins in relation to markers of endothelial function and inflammation. Eur J Epidemiol, 2004. 19(10): p. 915-21.
6. Engelhard, Y. N., B. Gazer, and E. Paran, Natural antioxidants from tomato extract reduce blood pressure in patients with grade-1 hypertension: a double-blind, placebo-controlled pilot study. Am Heart J, 2006. 151(1): p. 100.
7. Paran, E., et al., Effect of standartizised tomato extract on blood pressure, endothelial function and plasma lycopen in treated hypertensive patients. Am. J. Hypertens, 2005.

1. A method for lowering blood pressure in a subject, comprising the step of administering to said subject a blood pressure lowering effective amount of a composition comprising lycopene and at least one carotenoid.

2. A method of reducing vascular inflammation in a subject, comprising the step of administering to said subject an anti-inflammatory effective amount of a composition comprising lycopene and at least one carotenoid.

3. The method of claim 1 or 2, wherein the carotenoid is lutein.

4. The method according to claim 3, wherein the lycopene and the lutein together provide a synergistic therapeutic effect.

5. The method according to claim 4, wherein the synergistic combination of lycopene and lutein improves endothelial function by reducing the adhesion of neutrophils to cytokine-induced endothelial cells.

6. The method according to claim 1 or 2, wherein the lycopene is provided as a natural tomato extract.

7. The method according to claim 6, wherein the lycopene is extracted from tomato oleoresin.

8. The method according to claim 6, wherein the oleoresin further comprises at least one agent selected from the group consisting of tocopherols, beta-carotene, phytoene, phytofluene, phytosterols, tomato oil, and phospholipids.

9. The method according to claim 1 or 2, wherein the composition further comprises at least one agent selected from the group consisting of phytoene, phytofluene, phytosterols, zeaxanthin, astaxanthin, canthaxanthin and carnosic acid or a derivative thereof.

10. The method according to claim 1 or 2, wherein the composition is administered in combination with a conventional anti-hypertensive agent.

11. The method according to claim 1 or 2, wherein the composition is administered orally.

12. The method according to claim 11, wherein the composition is in a form selected from the group consisting of a tablet, caplet, capsule, microcapsule, pellet, pill, powder, syrup, gel, slurry, granule, suspension, dispersion, emulsion, liquid, solution, dragee, bead and beadlet.

13. The method of 12, wherein the composition is in the form of a soft gelatin capsule.

14. The method of claim 3, wherein the ratio of lycopene to lutein is about 1:10 to about 10:1.

15. The method of claim 14, wherein the ratio of lycopene to lutein is about 1:1.

16. The method of claim 3, wherein the amount of each of the lycopene and lutein is from about 1 mg to about 100 mg.

17. The method of claim 16, wherein the amount: of lycopene and lutein is about 15 mg and 20 mg, respectively.

18. A pharmaceutical composition for lowering blood pressure in a subject, comprising a blood pressure lowering effective amount of lycopene and lutein, wherein the ratio of lycopene to lutein is about 1:10 to about 10:1.

19. The composition of claim 18, wherein the ratio of lycopene to lutein is about 1:1.

20. The composition of claim 18, wherein the amount of each of the lycopene and lutein is from about 1 mg to about 100 mg.

21. The composition of claim 20, wherein the amount of lycopene and lutein is about 15 mg and 20 mg, respectively.

22. The composition according to claim 18, wherein the lycopene and the lutein together provide a synergistic therapeutic effect.

23. The composition according to claim 18, the synergistic combination of lycopene and lutein improves endothelial function by reducing the adhesion of neutrophils to cytokine-induced endothelial cells.

24. The composition according to claim 18, wherein the lycopene is provided as a natural tomato extract.

25. The composition according to claim 24, wherein the lycopene is extracted from tomato oleoresin.

26. The composition according to claim 25, wherein the oleoresin further comprises at least one agent selected from the group consisting of tocopherols, beta-carotene, phytoene, phytofluene, phytosterols, tomato oil, and phospholipids.

27. The composition according to claim 18, further comprising at least one agent selected from the group consisting of phytoene, phytofluene, phytosterols, zeaxanthin, astaxanthin, canthaxanthin and camosic acid or a derivative thereof.

28. The composition according to claim 18, further comprising one or more conventional anti-hypertensive agents.

29. The composition according to claim 18, formulated for oral administration.

30. The composition according to claim 29, wherein the composition is in a form selected from the group consisting of a tablet, caplet, capsule, microcapsule, pellet, pill, powder, syrup, gel, slurry, granule, suspension, dispersion, emulsion, liquid, solution, dragee, bead and beadlet

31. The composition of claim 29, in the form of a soft gelatin capsule.

32. Use of a composition according to claim 18 for the manufacture of a medicament for lowering blood pressure.

33. Use of a composition according to 18 for the manufacture of a medicament for reducing vascular inflammation.
